(19) United States
(12) Patent Application Publication
(10) Pub. No.: US 2009/0247575 A1
(43) Pub. Date: Oct. 1, 2009

(54) STABILIZING LIPID COMPOSITIONS FOR ORAL PHARMACEUTICAL AGENTS

(75) Inventors: Satish ASOTRA, Brampton (CA); Shen GAO, Bolton (CA); Avraham YACOBI, Englewood, NJ (US); Daniel A. MOROS, Larchmont, NY (US); Jerzy ZADYKOWICZ, Mississauga (CA)

Correspondence Address: TARO PHARMACEUTICALS U.S.A., INC.;C/O VENABLE LLP, P.O. BOX 34385, WASHINGTON, DC, 20043-9998, US

(73) Assignee: TARO PHARMACEUTICALS NORTH AMERICA, INC., Grand Cayman (KY)

(21) Appl. No.: 12/412,029

(22) Filed: Mar. 26, 2009

Related U.S. Application Data
(60) Provisional application No. 61/064,782, filed on Mar. 26, 2008.
(60) Provisional application No. 61/064,783, filed on Mar. 26, 2008.

(51) Int. Cl. A61K 31/47

(52) U.S. Cl. 514/311

(57) ABSTRACT

The present invention relates to a pharmaceutical composition for oral administration comprising an exceptionally labile active agent, a stabilizing vehicle comprising liquid triglycerides and a desiccant, wherein the composition is storage stable for an extended period without substantial degradation of the active agent.

This application claims the benefit of U.S. Provisional Appl. No. 61/064,872, filed Mar. 26, 2008, and U.S. Provisional Appl. No. 61/064,873, filed Mar. 26, 2008. Each of these applications is incorporated by reference in its entirety and for all purposes.

### FIELD OF THE INVENTION

The present invention relates to a pharmaceutical composition for oral administration comprising an exceptionally labile active agent, a stabilizing vehicle comprising liquid triglycerides and a desiccant, wherein the composition is storage stable for an extended period without substantial degradation of the active agent.

### BACKGROUND OF THE INVENTION

Many components in pharmaceutical formulations, including active agents, are chemically unstable. Degradation can occur in the presence of agents such as UV light, heat, oxygen, and/or water or moisture. As a result, many pharmaceutical formulations are restricted in the manner of their delivery. For example, some formulations require the dose to be in a solid dosage form such as a tablet or capsule, which are unsuitable for young children and other patients. Some solid dosage forms must be administered within minutes after opening the formulation's container, and any unused portion must be discarded after this time. Other formulations must be administered with a carrier, for example a small selection of soft foods. Such restrictions can lead to reduced patient compliance as well as wasted medication.

Montelukast is a leukotriene receptor antagonist that can be used in the treatment of asthma. Alsarra, I., Saudi Pharm. J. 12:4, 136-43. Without wishing to be bound by a particular theory, it is believed that montelukast works by binding to the cysteinyl leukotriene receptor CysLT₁, resulting in reduced bronchoconstriction and inflammation in lung tissue. It does not interact with theophylline, nor does it interact with many other asthma and allergy medications. See DrugBank (available at www.drugbank.ca). It has advantages over other asthma medications, for example corticosteroids, in that it can be administered orally, thus providing greater compliance.

Unfortunately, previous formulations containing montelukast suffer from rapid degradation. It is believed that degradation occurs in the presence of agents such as UV light, heat, oxidizing agents, and/or water or moisture and results in the formation of byproducts such as the corresponding sulfoxide. See, e.g., U.S. Pub. No. 2007/0184101. As a result, currently commercially-available montelukast formulations are either tablets or require the dose to be administered within 15 minutes after opening the container, and any unused portion has to be discarded after this time. In addition, the medication has to be administered with a carrier, and only a few carriers are permitted: cold or room temperature baby formula or breast milk for infants, or a spoonful of cold or room temperature applesauce, carrots, rice, or ice cream. Such restrictions can lead to reduced patient compliance as well as wasted medication.

Therefore, there exists a continuing need to develop vehicles that can stabilize exceptionally labile active agents, for example montelukast, as well as other components of a pharmaceutical composition.

### SUMMARY OF THE INVENTION

The inventive compositions, kits and methods disclosed herein meet the need for vehicles that can stabilize exceptionally labile active agents, such as montelukast sodium.

In one aspect, the invention provides pharmaceutical compositions for oral administration. The pharmaceutical compositions can comprise an exceptionally labile active agent, a stabilizing vehicle comprising liquid triglycerides and a desiccant. In some embodiments, the composition is storage stable for an extended period without substantial degradation of the active agent. The liquid triglycerides can comprise, for example, medium chain triglycerides. The active agent can be in suspension or in solution in the vehicle. In some embodiments, the triglycerides have a water content of less than about 0.01%. The active agent can be, without limitation, clindamycin, lansoprazole, alendronate, niaprazine, zafirlukast, pranlukast and/or montelukast, including derivative forms such as montelukast sodium. The compositions are storage stable for long periods of time, for example at least about 10 weeks, at least about 1 year, or at least about 2 years when stored at a temperature of about 20° C. to about 25° C.

In some embodiments, the pharmaceutical compositions comprise an exceptionally labile active agent in a stabilizing solid vehicle comprising a desiccant, wherein the composition is suitable for combining with a liquid vehicle comprising medium chain triglycerides. The active agent in these embodiments can be, for example, montelukast sodium, and the desiccant can be, for example, spray dried and/or granular mannitol. The composition can also comprise, for example, a silica gel, such as a colloidal silicon dioxide. The stabilizing vehicle can be in the form of granules, for example coated granules. The coating for the coated granules can provide greater stability at room temperature and can comprise a material including, for example, polyvinyl acetate phthalate, hydroxypropyl methylcellulose phthalate, a methacrylic acid-methacrylic acid ester copolymer, cellulose acetate trimellitate, carboxymethyl ethylcellulose, hydroxypropyl methylcellulose acetate succinate, a methacrylic acid-ethyl acrylate copolymer, a methacrylic acid-methyl methacrylate copolymer, a basic butylated methacrylate copolymer, an ammonio methacrylate copolymer, a poly(ethylacrylat-methylmethacrylat) dispersion, a methacrylic acid co-polymer, ethyl cellulose coating, and combinations thereof. In some embodiments, the granules or coated granules can be used in a reconstituted composition also comprising medium chain triglycerides. The medium chain triglycerides in the reconstituted composition can have a water content of less than about 0.01%. The reconstituted composition can also comprise an antioxidant.

In another aspect, the invention provides methods of making the compositions of the present invention. In some embodiments, the methods comprise the step of combining the exceptionally labile active agent with a vehicle comprising medium chain triglycerides and a desiccant. In other embodiments, the methods can comprise the steps of dissolving the exceptionally labile active agent in anhydrous ethanol to make a solution, and diluting the solution with a vehicle comprising dry medium chain triglycerides and a desiccant, thereby making the composition. In still other embodiments, the methods can comprise the steps of blending the exceptionally labile active agent with granular mannitol to make a powder blend, granulating the powder blend with a solution comprising hydroxypropylcellulose and anhydrous ethanol to make a granulation comprising particles, and drying the granulation and sizing the. particles. In yet other embodiments, the methods comprise combining granules or coated granules comprising an active agent and a desiccant with medium chain triglycerides.

In yet another aspect, the invention provides methods of treating a patient with the compositions of the present invention. In some embodiments, the methods comprise administering a therapeutically effective amount of one of the compositions of the present invention to the patient orally, thereby treating the illness. The patient can be, for example, a pediatric patient. The therapeutically effective amount of the composition can be, for example, about 5 mL.

In still another aspect, the invention provides kits. In some embodiments, the kit comprises a container and a composition comprising an exceptionally labile active agent, a vehicle comprising triglycerides and a desiccant. In some embodiments, the triglycerides have a water content of less than about 0.01%. The container can be, for example, a light-protective container. The vehicle can contain antioxidants such as BHA and BHT. The container can contain one dose or multiple doses of a therapeutically effective amount of the composition. The composition can be present in an amount of up to or at least about about 100 mL, about. 200 mL, about 250 mL, or more. The container can be made of, for example, high density polyethylene, and can comprise a closure device that can be removed and then replaced to reseal the container.

The kits can also comprise a first container comprising a therapeutically effective amount of a solid composition comprising an exceptionally labile active agent and a desiccant, and a second container comprising a pharmaceutically acceptable liquid vehicle comprising triglycerides, and an excipient, diluent or combination thereof, suitable for oral administration. Either or both of the first container and second container can be light-protective containers, and either or both can be made of high density polyethylene. The vehicle can also include antioxidants, such as, for example, BHA and/or BHT.

### DETAILED DESCRIPTION

Embodiments of the invention are discussed in detail below, using specific terminology for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

The headings below are provided solely for organizational purposes and are not intended to impart any division or meaning to this document, unless specifically indicated.

### Compositions

The present invention is directed to a storage-stable orally administrable pharmaceutical solution or suspension comprising an exceptionally labile active agent in a vehicle comprising triglycerides. In the compositions of the invention, the active agent is stable from degradation that would otherwise occur upon exposure to light, heat, oxidizing agents, or water. In a further embodiment, the invention comprises a desiccant. In another aspect, the invention comprises a desiccated triglyceride. In another embodiment, the desiccated triglyceride is desiccated medium chain triglyceride (MCT) oil. In some embodiments, the active agent is in solution in the vehicle. In some embodiments, the active agent is in suspension in the vehicle. The suspension can be formed from active agent-containing granules, which may be coated, and which may be mixed with a liquid vehicle comprising triglycerides in a reconstitution process. For example, the compositions of the present invention can comprise an MCT oil, a desiccant such as mannitol, anhydrous ethanol or anhydrous silicon dioxide, and an exceptionally labile active agent such as montelukast sodium. In some embodiments, the invention provides a liquid pharmaceutical composition for oral administration. In another embodiment, the active in the composition is stabilized by using an antioxidant or a light-protective container.

The term “active agent” encompasses any pharmaceutically acceptable compound that can alleviate the symptoms of an illness.

An “illness” is any disorder or condition that can be treated by pharmaceutical means. “Illness” encompasses disorders or conditions affecting any tissue, organ or organ system of the body. “Illness” includes any condition for which a physician might prescribe a medication comprising an active agent, as well as any condition for which a patient might seek an over-the-counter remedy. “Over-the-counter” means a medication or remedy that a patient can purchase without the authorization of a physician or other health care provider.

“Suspension” encompasses any combination of two substances in which one substance is mixed with but undissolved in another substance, for example a fluid or solid. “Suspension” also encompasses a system in which a solid is dispersed in a solid, liquid, or gas, for example in particles of larger than colloidal size.

“Solution” encompasses any system in which one substance is dissolved in another. The term can also be used to describe the process of preparing a solution.

“Labile” means unstable. “Labile” can refer to compounds subject to degradation, for example hydrolytic, oxidative, photochemical and/or thermal degradation. “Labile active agent” encompasses any pharmaceutically acceptable compound that can alleviate the symptoms of an illness and that is also subject to degradation, for example hydrolytic, oxidative, photochemical and/or thermal degradation. The term “active agent” encompasses “labile active agent,” such that “active agent” can be understood to include labile active agents.

“Labile active agent” can encompass exceptionally labile active agents. As used herein, “exceptionally labile active agents” encompasses active agents, such as montelukast, that undergo degradation over time when included in unmodified commercially-available MCT oils, but which exhibit long-term storage stability in desiccated MCT oils.

Without wishing to be bound by any particular theory, montelukast is exceptionally labile due to the presence of a cyclopropyl group in its structure. Three- and four-membered rings exhibit heightened instability, as predicted under Baeyer strain theory, and agents that include them are often exceptionally labile. Examples of compounds with three- or four-membered ring substituents include, without limitation, naltrexone, efavirenz, nevirapine, orbifloxacin, pitavastatin, ciprofloxacin, sparfloxacin, iclaprim, gemifloxacin, moxifloxacin, calcipotriol/calcipotriene, boceprevir and sibutramine. In addition, compounds that are exceptionally unstable for other reasons are also contemplated within the definition of “exceptionally labile active agent.”

Under the Baeyer strain theory, when carbon is bonded to four other atoms, the angle between any pair of bonds is the tetrahedral angle 109.5°. Deviations from this angle in cyclic (ring) compounds such as cyclopropane cause molecules to be strained and therefore relatively unstable. The greater the deviation from this angle the more unstable a molecule is and thus the more prone it is to ring-opening reactions. Based on this theory, stability of 3- and 4-member carbon rings is expected to be lower than that of 5-, 6- and larger member rings.

In some embodiments, the compositions of the invention can comprise more than one active agent. In some embodiments, the present invention comprises two, three, four, five or more active agents in combination. The active agents used in the embodiments of the invention that comprise more than one active agent or a mixture thereof.

In some embodiments, the invention relates to a system for inhibiting or preventing degradation of an exceptionally labile pharmaceutical ingredient in a composition, for example by using a vehicle comprising desiccated MCT oil.

Certain active agents can be susceptible to inactivation by degradation. Degradation can occur in the presence of agents such as UV light, oxygen, water and moisture, and heat.

Such degradation presents a major problem. For example, for some medications, a dose must be administered within minutes after opening the medication's container, and any unused portion must be discarded after this time. Other medications must be administered with a carrier, for example a small selection of soft foods. Such restrictions can lead to reduced patient compliance as well as wasted medication.

In a substantial and unexpected improvement, the compositions of the present invention are stable at room temperature for an extended period of time, up to about 2 weeks, or up to about 4, about 6, about 8 or about 10 weeks, about 3 months, about 4 months, about 6 months, about 1 year, about 2 years, about 3 years or longer. Such stability can be for a container kept unopened (i.e., sealed) since initial packaging, or for a container that is opened and then resealed after initial use, for example after dispensing a dose of the composition.

In some embodiments, the composition is in a vehicle comprising desiccated medium chain triglycerides (MCT) and forms stable lipid compositions for an active agent. In some embodiments, the MCT vehicle provides enhanced stability for one or more composition components other than the active agent. Various forms of lipid compositions were developed which are stable for extended periods at room temperature after preparation using the methods of preparation of the invention, e.g., anhydrous oral solution, stabilized oral granules for reconstitution, coated granules for extended stability at room temperature, and controlled/extended/slow release coated granules.

In some embodiments, the composition can be reconstituted before being administered to a patient. For example, embodiments of the invention can be reconstituted using triglycerides, including medium chain triglycerides. “Reconstitute” means to combine components of a formulation, such as a component with the active ingredient and a vehicle, to produce a pharmaceutically acceptable formulation. Reconstituted formulations are one form of the composition that can be administered to a patient.

In some embodiments, the invention relates to a pharmaceutical composition consisting essentially of an MCT oil and an active agent, and optionally an antioxidant and/or a dessicant. In some embodiments, the active agent is in the form of coated granules. In some embodiments, the invention provides methods of administering any of the compositions and systems of the present invention according, for example, to the methods of preparation and treatment disclosed herein.

The term “vehicle” includes any ingredient or combination of ingredients in a formulation other than the active ingredient. The “vehicle” is appropriately described as such whether the active agent or agents are included in the vehicle or not.

In some embodiments, the active agent is an asthma medication. The term “asthma medication” encompasses any pharmaceutically acceptable compound that can alleviate the symptoms of asthma, for example bronchoconstriction, inflammation, contraction of airway smooth muscle tissue, localized edema in airway tissue walls, mucus secretion in the airways, and increased airway resistance. This term includes, for example, leukotriene receptor antagonists. For example, the term “asthma medication” encompasses montelukast and all its derivatives, including acids, bases, salts, and esters thereof, as well as polymorph crystal forms. “Asthma medication” accordingly encompasses montelukast sodium. “Asthma medication” also includes medications effective against exercise-induced bronchoconstriction, also referred to as exercise-induced asthma or exercise-induced bronchoconstriction. “Asthma medication” also includes medications effective against allergies, including, for example, seasonal allergies. As used herein, the term “montelukast” encompasses montelukast and all its derivatives, including acids, bases, salts (including, for example, alkali metal salts such as sodium or potassium, alkaline earth salts or an ammonium salt), and esters thereof, as well as polymorph crystal forms and any other form of montelukast that has one or more of the pharmaceutical activities of montelukast. “Montelukast” accordingly encompasses montelukast sodium.

Suitable asthma medications for use in the present invention include, but are not limited to, the leukotriene receptor antagonists, which include zafirlukast, montelukast and pranlukast. These compounds are useful in the treatment of asthma. These compounds bind with leukotriene receptors, thereby reducing bronchoactive responses mediated by the receptors. Such bronchoactive responses include airway smooth muscle contraction, microvascular hyperpermeability, and mucus hypersecrection. Diamant. Z. et al., Clin Exp Allergy 29:42-51.

A “leukotriene receptor antagonist” is a compound that can bind to a leukotriene receptor, thereby inactivating it. The term encompasses antagonists that bind reversibly and irreversibly to the receptor. It also encompasses compounds that have the same effect as a leukotriene receptor antagonist, for example by directly or indirectly inhibiting 5-lipoxygenase pathways. As one of skill in the art will appreciate, this term encompasses all forms of the compound including, for example, acids, bases, salts, and esters thereof, as well as polymorph crystal forms. In some embodiments, a “leukotriene receptor antagonist” refers to a pharmaceutically acceptable salt selected from, but not limited to, alkali metal salts such as sodium or potassium, alkaline earth salts or an ammonium salt (all of which are herein referred to as a pharmaceutically acceptable salts). For example, the term includes montelukast sodium.

In another aspect, the leukotriene receptor antagonists can be effective against any disease or disorder that is mediated by leukotriene receptors according to the present invention. Such diseases or disorders include allergies, including seasonal allergies, exercise-induced bronchoconstriction, airway inflammation, and allergic rhinitis, as well as migraine and colitis.

The invention is further directed to using leukotriene receptor antagonists other than zafirlukast, montelukast, and pranlukast. As one of skill in the art will appreciate, the above list of active agents is not exhaustive and other active agents which are not mentioned are also encompassed within the present invention.

Montelukast is one example of a leukotriene receptor antagonist. Montelukast is a substituted quinoline compound that is freely soluble in ethanol and methanol. Its structure is presented below:

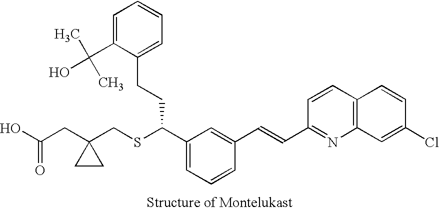

Leukotriene receptor antagonists such as, for example, montelukast, pranlukast and zafirlukast, can be extremely susceptible to inactivation by degradation. It is believed that degradation occurs in the presence of agents such as UV light, oxidizing agents, water and moisture, and heat and results in the formation of byproducts such as its corresponding sulfoxide.

Degradation presents a major problem for compositions comprising exceptionally labile active agents. For example, montelukast is exceptionally labile. It will degrade over time if formulated in an unmodified, commercially-available MCT oil. However, surprisingly, montelukast sodium is stable for extended periods of time, for example up to or at least about 2 years, when formulated in a desiccated MCT oil. The inventive compositions and methods avoid problems that can lead to reduced patient compliance as well as wasted medication.

Montelukast does not interfere with theophylline, nor does it interfere with many other asthma and allergy medications. The asthma medications used in the embodiments of the present invention that comprise more than one active agents can be selected from a group including, but not limited to, theophylline, loratadine, β-adrenergic agonists, corticosteroids, cromilyn and nedocromil, ipratropium, zafirlukast, pranlukast, zileuton, α-adrenergic agonists, cetirizine, dextromethorphan, guaifenesin, chlorpheramine, opiates, or a mixture thereof.

In some embodiments, montelukast can be formulated in coated granules, which can be suspended in a liquid vehicle comprising triglycerides, such as, for example, medium chain triglycerides. A reconstituted pharmaceutical composition is thus formed. Such reconstitution can be performed before the composition is administered to a patient. For example, embodiments of the invention can be reconstituted using triglycerides, including medium chain triglycerides.

According to the present invention, surprisingly, compositions comprising triglycerides, a desiccant and an active agent, for example an exceptionally labile active agent such as montelukast, prevents degradation of the active agent. It also prevents degradation of any component of the composition that is subject to degradation. Montelukast sodium is soluble in medium chain triglycerides at a concentration of, for example, approximately 3.5 mg/g.

Triglycerides (also known as triacylglycerol or triacylglyceride) are glycerides in which the glycerol is esterified with three fatty acids. Triglycerides are the main constituent of vegetable oil and animal fats. The general chemical structure of triglycerides is shown below:

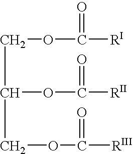

R′, R″, and R′″ are alkyl chains (C₁-Cₙ), and two or three may be the same or each may be different.

Chain lengths of the fatty acids in naturally occurring triglycerides may range from 3 to 24 carbon atoms, but lengths of 16 and 18 carbon atoms are most common. Shorter chain lengths may be found in some substances (for example, butyric acid in butter). Most naturally occurring fats contain a complex mixture of individual triglycerides. Based on their chain length, triglycerides can be divided into three categories: (i) short chain triglycerides (SCT); (ii) medium chain triglycerides (MCT); and (iii) long chain triglycerides (LCT).

Short chain triglycerides are triglycerides having short chain fatty acids, e.g., C₂-C₆. For example, one short chain triglyceride is glyceryl tributyrate.

Medium chain triglycerides have fatty acids ranging from about C₇ or C₈ to about C₁₀ or C₁₁. Some exemplary commercially available medium chain triglycerides are LABRAFAC® (available from Gattefossé Pharma, Saint-Priest Cedex, France) CAPTEX® (available from Parchem, White Plains, N.Y.), NESATOL® (available from Kreglinger Europe, Antwerp, Belgium) WAGLINOL® (available from Industrial Quimica Lasem, S.A., Barcelona, Spain), BERGABEST® (available from Sternchemie, Hamburg, Germany), MIGLYOL® (available from Universal Preserv-A-Chem, Inc., Edison, N.J.), NEOBEE® (available from Stepan Company, Northfield, Ill.), and CRODAMOL® (available from Croda, Edison, N.J.).

Triglycerides longer than a medium chain triglyceride, e.g. about C₁₁ and longer, are called long chain triglycerides. One example of an oil containing long chain triglycerides is castor oil.

The compositions of the present invention can comprise a short chain, medium chain, or long chain triglycerides, or combinations. In some embodiments, the triglyceride is a medium chain triglyceride. In some embodiments of the present invention, the use of a medium chain triglyceride can increase the bioavailability of a drug. In some embodiments, the triglyceride is a desiccated triglyceride, such as a desiccated medium chain triglyceride.

The present invention can also comprise a mixture of triglycerides. In some embodiments, the mixture of triglycerides comprises a mixture of short chain and medium chain, short chain and long chain, medium chain and long chain, or short chain, medium chain, and long chain triglycerides, which combination can be combined with a desiccant. Furthermore, the mixture of triglycerides can comprise triglycerides with more than one short chain, more than one medium chain, and/or more than one long chain. The triglycerides can also be heterogeneous. In heterogeneous triglycerides, one or more of the fatty acids in a single triglyceride may be of differing carbon chain lengths, including different lengths of medium-chain fatty acids or short-chain or long-chain fatty acids. Some or all of the chain lengths (i.e., R groups) may be C₅, C₆, C₇, C₈, C₉, C₁₀, C₁₁, or C₁₂. The chain lengths may be C₆ to C₁₁ or C₇ to C₁₀. Any of these triglycerides can be desiccated according to the invention. In addition, any liquid triglyceride composition, or any liquid combination of triglycerides or chain lengths within triglycerides recited above, can be used according to the invention.

The compositions of the present invention can comprise about 1% to about 99% triglycerides (w/w), or about 25% to 99% (w/w), about 75% to 99% (w/w), about 80% to 99% (w/w), or about 92% to 99% (w/w) triglycerides. In some embodiments, the inactive components of the present invention comprise about 1% to about 25%, about 25% to about 50%, about 50% to about 75%, or about 75% to about 100% triglycerides (w/w). In some embodiments, the composition comprises about 99%, about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5% of triglycerides (w/w). The term “about” generally refers to plus or minus 10% of the indicated number. For example, “about 10%” indicates a range of 9% to 11%. Other meanings of “about” may be apparent from the context.

In some embodiments, the triglycerides can be selected from the group including, but not limited to, vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides, and mixtures thereof.

In some embodiments, the triglycerides can be almond oil; babassu oil; borage oil; blackcurrant seed oil; canola oil; castor oil; coconut oil; corn oil; cottonseed oil; evening primrose oil; grapeseed oil; groundnut oil; mustard seed oil; olive oil; palm oil; palm kernel oil; peanut oil; rapeseed oil; safflower oil; sesame oil; shark liver oil; soybean oil; sunflower oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; partially hydrogenated soy and cottonseed oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; and glyceryl tricaprylate/caprate/stearate.

MCT oils suitable for use according to the invention may be clear or slightly yellowish esters of fatty acids (e.g., of saturated coconut and palm kernel oil-derived caprylic and capric acid) and glycerin or propylene glycol, with neutral odor and taste. Desirable properties include being very pure because of carefully selected raw materials and a controlled manufacturing process, containing few microorganisms and being free of additives or contaminants, such as antioxidants, solvents and catalyst residues. These oils have the following advantages in comparison to natural oils. They have high stability against oxidation, are liquid at 0° C., and have no undesirable effects on skin. They co-dissolve with many lipophilic solvents. They are quickly metabolized, and are not stored as body fat.

In some embodiments, the MCT oils of the invention are desiccated MCT oils. Desiccated MCT oils are dryer, i.e., contain less water, than commercially-available MCT oils. Desiccated MCT oils can be prepared by adding one or more water scavengers, or desiccants, to the MCT oil vehicle. Adding a water scavenger can substantially reduce the water content of unmodified commercially-available MCT oils.

Surprisingly, MCT oils can be formulated with pharmaceutical-grade desiccants, and such MCT oils comprising desiccants are capable of serving as a stabilizing vehicle for active agents, such as montelukast, that cannot be stored for long periods in unmodified, commercially-available MCT oils because they undergo degradation when exposed to the water present in unmodified MCT oils. Use of a desiccant in an MCT oil vehicle to stabilize such exceptionally water-labile active agents, especially those that are water-soluble such as montelukast sodium, was not contemplated previously.

In embodiments in which the MCT oil is desiccated, it is generally better to minimize the water content. Without wishing to be bound by any particular theory, it is believed that montelukast sodium reacts with water in a 1-to-1 molar ratio. Therefore, minimizing water content will serve to preserve a greater percentage of montelukast sodium. Preserving about 80, about 85, about 90, about 95 percent or more of the montelukast sodium originally included in the composition is generally consistent with storage stability. Thus, the water content of the MCT oil should be kept below that generally found in commercially available MCT oils. E.g., a water content of about 0.020% (w/w) or lower may be acceptable, as is a water content of about 0.015% (w/w), about 0.01% (w/w), about 0.005% (w/w), about 0.001% (w/w), about 0.0005% (w/w) or lower. MCT oils with these water contents are considered “desiccated” as this term is used herein.

The compositions of the present invention can utilize non-triglyceride agents in the vehicle. For example, the vehicle can comprise a desiccant or sorbent material such as mannitol. As used herein, “desiccant” encompasses any agent that, when added to a triglyceride vehicle, decreases its water content sufficiently to allow for stable storage of exceptionally labile active agents, such as montelukast sodium. All desiccants used in the compositions of the invention are pharmaceutical grade and conform to United States Pharmacopeia (“USP”) standards for excipients to be included in a pharmaceutical composition for oral administration. An example of such a desiccant is mannitol.

Mannitol is a sorbent material that is commonly used in dry, solid formulations. It can be used to produce a dry, free-flowing, granulated powder or powders that can provide an extended shelf life and be more convenient to handle and mix in the field. Mannitol comes in several forms, including spray dried and granular. Useful substitutes for mannitol include other polyhydric alcohols, such as sorbitol, xylitol, isomalt and malitol, as well as tribasic calcium phosphate, dibasic calcium phosphate, calcium phosphate, kaolin, lactose, microcrystalline cellulose, powdered cellulose, precipitate calcium carbonate, starch, dextrose, dextrate, sucrose, anhydrous silicon dioxide, anhydrous ethanol, sodium metabisulfite and mixtures thereof. See., e.g., U.S. Pat. No. 7,276,468. Sorbent materials and water scavengers can act as desiccants. As used herein, “water scavenger” and “desiccant” are substantially equivalent terms. Anhyrdous ethanol can act as a water scavenger or desiccant, for example through its formation of hydrate complexes with water molecules. An example of an anhydrous silicon dioxide is Syloid 244, a colloidal silicon dioxide.

In some embodiments, granular mannitol is present in an amount ranging from about 1% to about 99% (w/w), from about 10% to about 80% (w/w), from about 15% to about 60% (w/w), or from about 20% to about 40% (w/w). In some embodiments, granular mannitol can be present in an amount of about 5% (w/w), about 10% (w/w), about 15% (w/w), about 30% (w/w), about 35% (w/w), about 40% (w/w), about 45% (w/w), or about 50% (w/w) or more.

In some embodiments, spray dried mannitol is present in an amount ranging from about 10% to about 99% (w/w), from about 30% to about 90% (w/w), from about 40% to about 80% (w/w), or from about 50% to about 70% (w/w). In some embodiments, granular mannitol can be present in an amount of about 35% (w/w), about 40% (w/w), about 45% (w/w), about 50% (w/w), about 55% (w/w), about 60% (w/w), about 65% (w/w), about 70% (w/w), about 75% (w/w), about 80% (w/w), about 85% (w/w), or about 90% (w/w) or more.

Thus, in some embodiments, the compositions of the present invention comprise an MCT oil, a desiccant such as mannitol, anhydrous silica gel or anhydrous ethanol, and an exceptionally labile active agent such as montelukast sodium. For example, the composition can comprise about 94% (w/w) Miglyol® 812, about 5% (w/w) anhydrous ethyl alcohol, about 0.01% (w/w) BHA, about 0.01% (w/w) BHT, and about 0.395% (w/w) montelukast sodium. The MCT oil become a desiccated MCT oil by virtue of the addition of a desiccant to the composition.

In some embodiments, the compositions of the present invention comprise granules comprising a desiccant such as mannitol and an exceptionally labile active agent such as montelukast sodium. The granules can be coated with, for example, an enteric coating. The granules or coated granules can be combined with an MCT oil to form a suspension of granules or coated granules in MCT oil. For example, the granules can comprise about 30% (w/w) mannitol granular, about 64.88% (w/w) mannitol spray dried, about 3% (w/w) Syloid 244, about 1.7% (w/w) hydroxypropyl cellulose, and about 0.42% (w/w) montelukast sodium. These granules can be combined with sufficient MCT oil to make a composition with an active agent concentration of 4 mg/mL.

In some embodiments, the compositions of the present invention comprise a suspension of montelukast sodium in desiccated MCT oil. For example, the composition can comprise about 98.5% (w/w) Miglyol® 812, about 1% (w/w) Syloid 244 silica gel, about 0.395% (w/w) montelukast sodium, about 0.01% (w/w) BHA and about 0.01% (w/w) BHT. In this composition, the Syloid 244 acts both as a dispersant and as a desiccant.

In some embodiments, the compositions of the present invention (e.g., solid preparations of the active component for reconstituting with triglycerides, or after reconstitution) further comprise enteric coatings on the. active component. An “enteric coating” is a barrier applied to oral medication that controls the location in the digestive system where it is absorbed. For example, enteric coatings can prevent release of medication until it reaches the small intestine. Examples of such enteric coatings include polyvinyl acetate phthalate, hydroxypropyl methylcellulose phthalate, acrylic copolymers such as methacrylic acid-methacrylic acid ester copolymers, cellulose acetate trimellitate, carboxymethyl ethylcellulose, and hydroxypropyl methylcellulose acetate succinate. See, e.g., Remington: The Science and Practice of Pharmacy, 21^{st} Ed. (2006). Other examples include EUDRAGIT® products L100-55 (comprising methacrylic acid-ethyl acrylate copolymer), L100 (comprising methacrylic acid-methyl methacrylate copolymer in a ratio of about 1:1), S100 (comprising methacrylic acid-methyl methacrylate copolymer in a ratio of about 1:2), E100 (comprising a basic butylated methacrylate copolymer), RL100 (comprising an ammonio methacrylate copolymer), RS100 (comprising an ammonio methacrylate copolymer), and NE 30 D (comprising a poly(ethylacrylat-methylmethacrylat) dispersion), all from Evonik Röhm GmbH, Darmstadt, Germany; Sureteric® (comprising polyvinyl acetate phthalate), Acryl-EZE® (comprising a methacrylic acid co-polymer) and Coateric® (comprising polyvinyl acetate phthalate), all from Colorcon, West Point, Pa.; and ethyl cellulose coating. The enteric coating can also comprise any other suitable enteric coating known in the art. The enteric coating can also comprise two or more of the listed coatings as well as any other suitable enteric coatings.

In some embodiments, the pre-constituted or reconstituted compositions of the present invention can be coated and conjugated to produce a controlled/slow/extended release drug formulation. Examples of such coatings include: mixtures of waxes (for example, beeswax, carnauba wax) with glyceryl monostearate, stearic acid, palmitic acid, glyceryl monopalmitate, and/or cetyl alcohol; shellac and zein; ethylcellulose; acrylic resins and other acrylic polymers; cellulose acetate; silicone elastomers; polyvinyl acetate phthalate; and combinations thereof. See, e.g., Remington: The Science and Practice of Pharmacy, 21^{st} Ed. (2006). As will be appreciated by one of skill in the art, any suitable coating or conjugation technique that provides controlled/slow/extended release characteristics are also encompassed within the present invention.

Generally, in preparing formulations in which the desiccant is included in the MCT oil, the desiccant is added to the MCT oil before adding the active agent. However, the order of addition is not critical, so long as it is consistent with minimizing the degradation of the exceptionally labile active agent.

Without wishing to be bound by any particular theory, it is believed that MCT oil, if left exposed to a water source such as, for example, air, will increase in water content over time. Adding a desiccant can serve to minimize or avoid this increase, keeping the water content below about, for example, 0.03% (w/w).

The compositions of the present invention have the unexpected advantage of being stable for an extended period, for example when stored at room temperature, i.e., a temperature of about 20° C. to about 25° C. In some embodiments, the compositions of the present invention are stable for about 2 weeks, about 4 weeks, about 6 weeks, about 8 weeks, or about 10 weeks, about 3 months, about 6 months, about 9 months, about 1 year, about 2 years, about 3 years or indefinitely when stored at a temperature of about 20° C. to about 25° C.

In compositions comprising montelukast sodium, the montelukast sodium can be present in an amount from about 0.01 to about 10% (w/w), or about 0.1% to about 5% (w/w), or about 0.1% to about 1.0% (w/w). In some embodiments, the montelukast concentration is about 0.01%, about 0.05%, about 0.1%, about 0.15%, about 0.2%, about 0.25%, about 0.3%, about 0.35%, about 0.4%, about 0.45%, about 0.5%, or about 0.14% (w/w), or about 0.395% (w/w), or about 0.42% (w/w). However, the precise dosage can be varied and still be within the scope of the present invention, as would be appreciated by a person of ordinary skill in the art.

The dosage amount of the final formulation (e.g., after reconstitution in the case of granules or coated granules) can similarly be varied and still be within the scope of the present invention. For example, the dosage amount can be in the range from about 0.5 to about 20 ml, or about 1 to about 10 ml, or about 3 to about 7 ml. For example, the dosage amount can be about 1, about 2, about 3, about 4, about 5, about 6, about 7, about 8, about 9, about 10 ml or more. Generally, the dosage can be administered once per day, but other administration regimens are also contemplated. For example, the dosage can be administered 4 times per day, 3 times per day, 2 times per day, once per day, or once every 2 days, 3 times per week, 2 times per week, or once per week, or irregularly, e.g., on an as-needed basis.

The composition can be packaged by any means known in the art that is consistent with avoidance of degradation of the exceptionally labile active agent. For example, the composition can be packaged in a bottle.in a manner similar to over-the-counter. cough medications. For example, the composition can be delivered in a ready-for-reconstitution form, in which granules are stored in one container (e.g., a pouch or bottle) and the MCT oil is stored in a separate container, for reconstitution by a pharmacist, the user, or other person.

Embodiments of the present invention are directed to compositions that maintain desirable organoleptic properties for extended periods of time. Organoleptic properties can include the color, smell, texture, taste, feel and other properties related to the overall palatability of the composition. The improved storage profiles of the compositions of the present invention prevent settling, discoloration, and other changes in the composition during storage for extended periods of time. This can allow the compositions to maintain desirable organoleptic properties for longer periods of time than would otherwise be expected.

The present invention can further comprise a pharmaceutically acceptable excipient other than the triglycerides or solid vehicles previously mentioned. The excipient may be mixed or dissolved directly in the triglyceride liquid, or it may be combined with the active agent in solid or liquid form and then reconstituted. An “excipient” refers to a substance that is used in the formulation of pharmaceutical compositions, and, by itself, generally has little or no therapeutic value. Various excipients can be used in the invention, including those described in Remington: The Science and Practice of Pharmacy, 21^{st} Ed. (2006). Excipients include, but are not limited to, antioxidants, anti-bacterial agents that prevent the decay of the formulation itself as opposed to those exhibiting a therapeutic effect, preservatives, chelating agents, buffering agents, agents for adjusting toxicity, colorings, flavorings and diluting agents, emulsifying and suspending agents, solvents, dispersing agents, binders, disintegrants, and other substances with pharmaceutical applications. Such excipients can include, for example, anhydrous ethyl alcohol, butylated hydroxyanisole (BHA), butylated hydroxytoluene (BHT), silica gel, for example a colloidal silicium dioxide such as SYLOID® 244 FP silica gel (Grace Davison, Baltimore, Md.), and hydroxypropyl cellulose (including KLUCEL LF).

The term “pharmaceutically acceptable” vehicle, agent and excipient, as well as dosage, refer to those compounds, materials, compositions, and/or dosage forms which are, within the scope of sound medical judgment, suitable for contact with the tissues of human beings and animals without excessive toxicity, irritation, allergic response, or other problems or complications commensurate with a reasonable benefit/risk ratio. In some embodiments, the term “pharmaceutically acceptable” can mean approved by a regulatory agency of the Federal or a state government or listed in the U.S. Pharmacopoeia or other generally recognized international pharmacopoeia for use in animals, and more particularly in humans. In some embodiments, the excipients used in the compositions of the present invention are pharmaceutically acceptable for oral administration.

In some embodiments, the invention provides a system for protecting an unstable active agent or other component against degradation, e.g., hydrolytic, oxidative, and/or photochemical degradation, with the resulting advantage of storage-stability for active agents or other components that otherwise would have a limited shelf life. The inventive system also unexpectedly provides ready absorption of the active agent after administration to the patient, and a palatable formulation. The system can include a light-protective container, a vehicle comprising an oil, for example a desiccated MCT oil, and/or a coating applied to granules of the active agent.

“Light-protective container,” also referred to as “light protective kit,” encompasses any container that inhibits or excludes entry of any electromagnetic radiation that would cause degradation of the active agent or other component held within the container. Such electromagnetic radiation includes, for example, visible light, ultraviolet (UV) radiation, infrared radiation, microwaves and radio waves. In some embodiments, the light protective container is opaque—i.e., it excludes most or all light. Examples of opaque containers include, but are not limited to, certain kinds of plastic medicine bottles or vials, e.g., those made of HDPE, and foil packets. In some embodiments, the light-protective container is translucent but colored in such a way as to exclude or inhibit entry of most light. Examples of such translucent light-protective containers include, but are not limited to, amber, dark brown or dark blue glass bottles. In some embodiments, the light-protective container can be coated in such a way as to exclude or inhibit entry of only that electromagnetic radiation which would cause degradation of the particular active agent or component packaged in the light-protective container. Examples of such coated light-protective containers include, but are not limited to, HDPE bottles that incorporate UV absorbers or fluorescent dyes (also known as f-dyes), for example as described in International Patent Publication WO/2001/085568. Light protective containers can comprise one layer or many layers.

In some embodiments, the light-protective bottle can inhibit or prevent degradation of a photo-labile ingredient. “Photo-labile ingredient” encompasses any active agent or other component that is subject to degradation by photochemical means, including exposure to electromagnetic radiation other than visible light. Such a bottle can also provide protection against exposure to moisture and/or oxidizing agents, for example those in the air.

In some embodiments, the oil vehicle can protect a hydrolysis- or oxidation-labile active agent or other component against exposure to moisture and/or oxidizing agents. “Hydrolysis-labile ingredient” encompasses any active agent or other component that is subject to degradation by hydrolytic means, including exposure to water in aqueous solution and/or airborne moisture. “Oxidation-labile ingredient” encompasses any active agent or other component that is subject to degradation by oxidative means, including exposure to oxidizing agents in the vehicle and/or in the air. Examples of oxidizing agents include, but are not limited to, hypochlorite and other hypohalite compounds, iodine and other halogens, chlorite, chlorate, perchlorate, and other analogous halogen compounds, permanganate salts, ammonium cerium(IV) nitrate and other Cerium(IV) compounds, peroxide compounds, hexavalent chromium compounds such as chromic and dichromic acids and chromium trioxide, pyridinium chlorochromate (PCC), and chromate/dichromate compounds, peroxide compounds, Tollen's reagent and other silver compounds, copper compounds, iron(III), (V), (IV), ruthenium (III), (IV) compounds, sulfoxides, N-oxides, persulfuric acid, osmium tetroxide (OsO4), nitric acid, nitrous oxide free radicals, oxygen and ozone. Organic oxidizing agents include N-bromosaccharin, N-bromosuccinimide, N-tert-butylbenzenesulfinimidoyl chloride, tert-butyl hydroperoxide, 3-chloroperoxybenzoic acid, cumene hydroperoxide, 1,3-dibromo-5,5-dimethylhydantoin and other halide derivatives (DBDMH), formic acid, hydrogen peroxide urea adduct, iodobenzene halides and derivatives containing hypervalent iodine, and pyridinium compounds containing halogens.

Without wishing to be bound by any particular theory, one way in which the oil vehicle provides such protection is by providing a physical barrier that inhibits or prevents exposure of the active agent or other component to water or oxidizing agents in an aqueous environment and/or moisture or oxidizing agents in the air. This protection is surprisingly enhanced by inclusion of a desiccant, which unexpectedly allows MCT oils to be used as stabilizing vehicles for exceptionally labile active agents such as montelukast. MCTs can also contain double bonds, which act as natural scavengers against oxidizing agents. The oil vehicle can optionally include other antioxidants to provide further protection against oxidizing agents.

Inclusion of a desiccant in an MCT oil base has surprising advantages over other methods of removing water, such as passing the oil through ceramic molecular sieves. For example, an MCT oil dried through use of ceramic molecular sieves will regain the water removed during sieving if it is exposed to a water source, such as the air, through equilibration processes. An MCT oil desiccated by inclusion of a desiccant does not regain water content in this way.

MCT oil has numerous advantages over other oils as a vehicle according to some embodiments of the invention. These vehicles have desirable pharmacokinetic properties of absorption, digestion, distribution and excretion without toxicity. The pharmaceutics are robust enough to support a variety of different formulations. The vehicles do not interfere with the pharmacological efficacy of the active agents. They readily form stable solutions for lipid-soluble active agents. Surprisingly, these formulations provide a platform for liquid dosing of drugs which previously could only be formulated in dry, solid dosage forms.

MCT oils have several other advantages over other triglycerides. MCT oils have good quality control and are more uniform in their low water content across batches. Short chain triglycerides may exhibit a wider variability in water content than MCTs. Long chain triglycerides are generally solid at room temperature and tend to become rancid via an oxidative process. Thus, shorter chain and longer chain triglycerides are not as well-suited for pharmaceutical compositions for oral administration as medium chain triglycerides (e.g., those with chain length of 5-11 or 7-10).

The MCTs as used in the embodiments of this invention should be of pharmaceutical grade, and should be dry, i.e., free or nearly free of water. This dryness can be accomplished by inclusion of a desiccant such as mannitol or silica. While short-chain triglycerides may exhibit greater variability in water content than MCTs, different MCT oils can also exhibit variability in water content. For example, some grades of MCT oil have water content up to 2% (w/w). This variability in water content is acceptable for most applications for which MCT oils are used, including topical, cosmetic, and nutritional applications.

However, when MCT oils are used in the orally-administrable pharmaceutical compositions of the present invention, this wide variability is not acceptable. Water content can be controlled and/or reduced by including a desiccant in the composition. A vehicle can be described as “desiccated” if its water content is less than or equal to about 0.02%. For example, a “desiccated MCT oil” is an MCT oil with a water content of less than about 0.02% (w/w). “Desiccated MCT oil” and “desiccated medium chain triglycerides” are to be understood as substantially equivalent terms. Other triglyceride compositions, for example other liquid triglyceride compositions, can also be desiccated according to the invention.

Samples of MCT oils, e.g. from different manufacturing sources or different batches, can be tested for their water content and thus their suitability for use in the orally-administrable pharmaceutical formulations of the present invention. For example, water content can be evaluated using the Karl Fischer method, also referred to as Karl Fischer titration, either before of after addition of a desiccant. Once tested, MCT oil with the requisite low water content can be included in the orally-administrable pharmaceutical preparations, while samples or batches with excessive water content can be excluded from the orally-administrable pharmaceutical formulations.

The water content of several commercially-available MCT oils is provided in the table below:

| Sample | Lot # | Water content. % |
|---|---|---|
| Captex 355 | JF-1-122A | 0.075 |
| Captex 355 | JF-1-122B | 0.12 |
| Captex 355 | JF-1-122C | 0.14 |
| Fractionated Coconut oil | RM070191 | 0.070 |
| Miglyol | RM060349 | 0.034 |

Thus, the invention utilizes a triglyceride that can be provided consistently in dry batches with a desiccant, is resistant to oxidation, is liquid at room temperature, and has other desirable characteristics.

Liquid triglyceride compositions for oral administration can be superior to gel caps and parenteral administration for many applications. For example, gel caps are not suitable for administration to many pediatric patients because their oropharyngeal musculature is not sufficiently developed to swallow gel caps safely. “Pediatric” encompasses any patient who is of a suitable age to be treated by a pediatric physician. Parenteral administration is not suitable for daily self-administration by most patients and is frequently painful.

MCT oil is also very easily absorbed in the GI tract, and thus can improve the absorption of many active agents. It does not affect the lipid profile of the blood and does not cause diarrhea in the dosages of the present invention. There are few allergies to MCT oils, in contrast to many nut-based oils. MCTs are also absorbed intact into the bloodstream, in contrast to long-chain triglycerides, which must be catabolized prior to absorption. Accordingly, MCTs can be used as a vehicle in patients who have difficulty metabolizing normal fats, such as AIDS patients and those with pancreatic insufficiency. See, e.g., Caliari S et al., Medium chain triglyceride absorption in patients with pancreatic insufficiency. Scand. J. Gastroenterol. 1996, 31:90-94. MCTs have been demonstrated to be safe when consumed at a level up to 50% of total dietary fat. See, e.g., Traul K A et al., Review of the toxicologic properties of medium-chain triglycerides. Food Chem Toxicol. 2000, 38:79-98. Newborn babies may be fed formulations containing MCTs such as coconut oils, for example as a nutritional supplement.

In some embodiments, the active agent or other component can be coated with a coating that can inhibit or prevent hydrolytic, oxidative or photochemical degradation. Coatings are often used to provide controlled, delayed or sustained release of an active agent, either by delaying release of the active agent until it reaches the small intestine (as with many enteric coatings) or by modulating the rate of release of the active agent by any of a number of mechanisms. In a surprising and substantial improvement, such coatings can be modified and/or employed so as to inhibit or prevent hydrolytic, oxidative and/or photochemical degradation of an active agent, while also providing ready absorption in the GI tract of the patient. Without wishing to be bound by any particular theory, the coating can operate to provide a physical barrier against exposure to moisture, oxidizing agents and/or light. The granules can also include a sorbent agent. These agents can act as dessicants to provide further protection against hydrolysis. Examples of such sorbent agents include mannitol, sorbitol, xylitol, isomalt and malitol, as well as tribasic calcium phosphate, dibasic calcium phosphate, calcium phosphate, kaolin, lactose, microcrystalline cellulose, powdered cellulose, precipitate calcium carbonate, starch, dextrose, dextrate, sucrose, sodium sulfate, magnesium sulfate, and mixtures thereof.

In some embodiments, the coatings of the invention provide ready absorption once the composition reaches the GI tract. Coatings according to some embodiments of the invention break down at the prevailing pH of the stomach, for example around pH 1.5-3. In some embodiments, the invention provides a pharmaceutical composition that produces a stable ultra water-sensitive active agent. In some embodiments, the invention provides an ultra dry liquid vehicle comprising triglycerides.

“Thermo-labile” encompasses any active agent or other component that is subject to degradation by thermal means, including exposure to thermal energy from the surrounding environment and that generated by chemical reactions in the composition. Embodiments of the present invention can be useful for protecting thermo-labile active agents.

### Methods of Treating

The compositions of the present invention can be administered to any mammal in need of the composition that can experience the beneficial effects of the compounds of the invention. Any such mammal is considered a “patient.” Such patients include humans and non-humans, such as pets and farm animals. Accordingly, the present invention is directed to methods of treating an illness in a patient in need thereof comprising administering a composition comprising an active agent in a vehicle comprising triglycerides and/or mannitol to the patient.

Various patients can be treated with the compositions of the present invention. In some embodiments, the patient is a child, e.g., an infant, toddler, or another person younger than 12, 18 or 21. In some embodiments, the patient is about 55 years of age, or 65, or 75, or older.

The dosage of the active agent administered will be dependent upon the age, health, and weight of the recipient, kind of concurrent treatment, if any, the frequency of treatment, and the nature of the effect desired. The compositions of the present invention can contain a quantity of a compound(s) according to this invention in an amount effective to treat the condition, disorder or disease of the patient being treated. One of ordinary skill in the art will appreciate that a method of administering pharmaceutically effective amounts of the active agent to a patient in need thereof can be determined empirically, or by standards currently recognized in the medical arts. It will be understood that, when administered to, for example, a human patient, the total daily dosage of the agents of the compositions of the present invention will be decided within the scope of sound medical judgment by the attending physician.

The therapeutically effective dose for each patient will depend upon a variety of factors: the type and degree of the cellular response to be achieved; activity of the specific agent or composition employed; the specific agents or composition employed; the age, body weight, general health, gender and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

In some embodiments, the compositions of the present invention can be administered in combination with another therapeutic agent. Accordingly, the compositions of the present invention can also include one or more additional therapeutic agents such as, but not limited to, hydrophilic drugs, hydrophobic drugs, agents for topical administration, hydrophilic macromolecules, cytokines, peptidomimetics, peptides, proteins, toxoids, sera, antibodies, vaccines, nucleosides, nucleotides, nucleoside analogs, genetic materials and/or combinations thereof.

Additional examples of therapeutic agents that can be used in the pharmaceutical compositions of the present invention include, but are not limited to, other antineoplastic agents, analgesics and anti-inflammatory agents, anti-anginal agents, antihelmintics, anti-arrythmic agents, anti-arthritic agents, anti-microbial agents such as nifurprazine, antibiotic agents such as clindamycin, anti-viral agents, anti-coagulants, anti-depressants, antidiabetic agents, anti-epileptic agents, anti-emetics, anti-fungal agents including oral anti-fungal agents effective against internal fungal infestations, anti-gout agents, anti-hypertensive agents, anti-malarial agents, antimigraine agents, anti-muscarinic agents, anti-neurodegenerative agents such as huperzine, anti-Parkinson's agents, anti-protozoal agents, anti-thyroid agents, thyroid therapeutic agents, anti-tussives, anxiolytic agents, anti-asthma medications such as montelukast and also zafirlukast and pranlukast, hypnotic agents, neuroleptic agents, beta-blockers, cardiac inotropic agents, corticosteroids, diuretics, gastrointestinal agents, histamine H₂-receptor antagonists, immunosuppressants, keratolytics, lipid regulating agents, muscle relaxants, nutritional agents, osteoporosis medications such as alendronate, cytokines, peptidomimetics, peptides, piperazines, proteins, proton pump inhibitors such as lansoprazole, toxoids, sera, sedatives such as niaprazine (a histamine H1-receptor antagonist with sedative properties), sex hormones, sex hormone antagonists or agonists, stimulants antibodies, vaccines, nucleosides, nucleoside analogs and genetic materials. Amphiphilic therapeutic agents and nutritional agents can also be included.

The compositions surprisingly effectively reduce degradation of the active agent, e.g., due to hydrolysis, oxidation or photolysis. Hydrolytic degradation generally involves the addition of water across one or more bonds in a compound, resulting in the breakdown of the compound into two or more separate products. Compounds that are subject to hydrolytic degradation include esters, imides, amides, lactams, and those with azomethine or imine bonds. Active agents that are susceptible to hydrolysis include cocaine, physostigmine, aspirin, tetracaine, procaine, methyldopa, amobarbital, procaine, diazepam, penicillins, cephalosporins, barbiturates and benzodiazepines. See Remington: The Science and Practice of Pharmacy, 21^{st} Ed. (2006).

Oxidative degradation generally involves the breakdown of compounds upon exposure to oxidizing agents or free-radical generating compounds. Upon exposure to atmospheric oxygen some compounds undergo auto-oxidation, which generates highly reactive free-radicals that participate in self-perpetuating autocatalytic reactions. Free radical-absorbing or -reacting agents can be added to pharmaceutical preparations to protect pharmaceuticals that are susceptible to oxidative degradation. Such agents may operate by terminating the propagation of the chain reaction to form relatively stable, resonance-stabilized free radicals, or by forming products that are not free radicals. Compounds that are subject to oxidative degradation include phenols, aromatic amines, aldehydes, ethers, and unsaturated aliphatic compounds. Active agents that are susceptible to oxidative degradation include epinephrine, ascorbic acid, phenothiazine, and vitamin A. Unsaturated fatty acids (e.g., oleic acid) can also undergo oxidation, converting carbon-carbon double bonds into single bonds. See Remington: The Science and Practice of Pharmacy, 21^{st} Ed. (2006).

Photochemical degradation generally involves light-catalyzed oxidation-reduction reactions. Such degradation often involves free radical intermediates and occurs in mechanistically complex reactions. Compounds that are sensitive to photochemical degradation include riboflavin, nifedipine, and phenothiazine. See Remington: The Science and Practice of Pharmacy, 21^{st} Ed. (2006).

The terms “treat” and “treatment” refer to both therapeutic treatment and prophylactic or preventative measures, wherein the object is to prevent, slow the progression of, or lessen an undesired physiological condition, disorder or disease, or obtain beneficial or desired clinical results. For purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the condition, disorder or disease progression; amelioration of the condition, disorder or disease state; remission, whether partial or total, whether detectable or undetectable; or enhancement or improvement of the condition, disorder or disease. Treatment includes eliciting a clinically significant response, without excessive levels of side effects. Treatment also includes prolonging survival as compared to expected survival if not receiving treatment. In some embodiments, the present invention is directed to a method of treating an illness in a patient comprising administering a composition comprising an active agent in a vehicle comprising triglycerides to the patient in need thereof.

A “pharmaceutically effective amount” means an amount effective to provide a therapeutic effect during a period of treatment. The effect can be the treatment of an illness or a related condition. In some embodiments, the present invention is directed to a pharmaceutically effective amount of an active agent, as administered to a patient in need thereof. Treatment may be for an acute or a chronic condition, and the effective amount may be a single dose or multiple doses over a period of a day, days, weeks, months, or longer, as would be apparent to those skilled in the art.

### Kits

The present invention is also directed to kits comprising one or more compositions of the present invention. In some embodiments, the kits of the present invention comprise a container or other means for holding the compositions of the present invention. In some embodiments, there is one container, two containers, three containers, or more than three containers. In some embodiments, the kit comprises a container comprising a therapeutically effective amount of the composition and a pharmaceutically acceptable carrier, excipient, diluent or combination thereof. In some embodiments, the kits comprise a light-protective container. In some embodiments, the container comprises a closure device that can be removed and then replaced to reseal the container.

In some embodiments, the kit comprises (a) a first container or other means for containing a therapeutically effective amount of the composition of the present invention, and (b) a second container or other means for containing a pharmaceutically acceptable carrier, excipient, diluent or combination thereof. Optionally, the kit can have additional containers or other means for containing comprising a therapeutically effective amount of additional agents. In some embodiments, the kit comprises a first container comprising a therapeutically effective amount of an active agent, and a second container comprising a vehicle comprising triglycerides.

In some embodiments, the kit comprises a container or other means for containing for the separate compositions, such as, a divided bottle or a divided foil packet; however, the separate compositions can also be contained within a single, undivided container. Typically, the kit contains directions for administration of the separate components. The kit form is particularly advantageous when the separate components are administered at different dosage intervals, or when titration of the individual components of the combination is desired by the prescribing physician.

In some embodiments, the active agent is in one container or other means for containing and the solution comprising triglycerides is in another. The physician or pharmacist can then mix the two components to form a composition comprising an active agent in a vehicle comprising triglycerides. The pharmacist may have a stock container of triglycerides, and may dispense a specific volume into a container and combine it with the active agent. The container may be a plastic bottle in which the active agent is stored separately from the oil.

In some embodiments, the kit of the present invention further comprises an additional container or means for containing comprising a therapeutically effective amount of an agent selected from the group consisting of hydrophilic drugs, hydrophobic drugs, hydrophilic macromolecules, cytokines, peptidomimetics, peptides, proteins, toxoids, sera, antibodies, vaccines, nucleosides, nucleotides, nucleoside and/or nucleotide analogs, genetic materials and combinations thereof.

In some embodiments, the container or other means for containing of the kit is a bottle. This bottle can be moisture-proof, including a moisture proof cap. It is also important to select a bottle that will not be permeable to the triglycerides of the present invention, in particular to the medium chain triglycerides. Some polymers that are suitable for aqueous solutions would not be suitable for use in the kits of the present invention.

In some embodiments, the kit comprises printed labeling instructions. The printed labeling may provide instructions for administering any of the compositions, using any of the kits, or performing any other method herein described. The composition will be contained in any suitable container capable of holding and dispensing the dosage form and which will not significantly interact with the composition. The labeling instructions will be consistent with the methods of treatment described herein. The labeling may be associated with the container by any means that maintain a physical proximity of the two; by way of non-limiting example, they may both be contained in a packaging material such as a box or plastic shrink wrap or may be associated with the instructions being bonded to the container such as with glue that does not obscure the labeling instructions or other bonding or holding means. Such labeling can provide instructions for, for example, dispensing and administering the formulation orally by teaspoon.

Suitable containers or other means for containing include, but are not limited to, bottles made of high density polyethylene (HDPE), polypropylene (PP), glass, and metal. HDPE bottles are particularly suited for the present invention because molecules of HDPE have fewer branches and side chains, leading to higher density and smaller pores. This makes it an effective barrier to contain medium chain triglycerides within the bottle as well as an effective barrier to prevent an influx of water or moisture from the air, as well as blocking light and preventing influx of oxygen or other oxidants, Accordingly, in some embodiments, the container is a HDPE bottle.

As one of skill in the art will appreciate, the container is not limited to HDPE bottles. The following table shows a comparison of the properties of some bottles contemplated for use in the present invention.

| | Bottle | | | | |
|---|---|---|---|---|---|
| | PET | PETG | HDPE | LDPE | PP |
| Moisture | Fair to | Fair to Good | Good to | Good | Good to |
| barrier | Good | | Excellent | | Excellent |
| Oxygen | Good | Good | Poor | Poor | Poor |
| barrier | | | | | |
| Clarity | Clear | Clear | Opaque | Opaque | Translucent |

In some embodiments, the present invention is directed to methods of administering an active agent to a patient. In some embodiments, the methods of administration comprise dispensing a therapeutically effective dose from a kit of the present invention into a receptacle, and delivering the dose from the receptacle to the patient. In some embodiments, the methods comprise administering an active agent to a patient, comprising causing a therapeutically effective dose to be delivered to the patient from a kit of the present invention.

In some embodiments, the present invention is directed to methods of preparing a ready-to-use asthma medication, comprising combining the active agent in the first container of a kit of the present invention and the vehicle of the second container of a kit of the present invention to form a supply of a ready-to-use asthma medication.

In some embodiments, the methods of administering comprise combining the composition in the first container of a kit of the present invention and the carrier, excipient, diluent or combination thereof of the second container of the kit of the present invention to form a therapeutically effective pharmaceutical preparation, and causing a therapeutically effective dose of the pharmaceutical preparation to be delivered to the patient. In some embodiments, the methods of administration comprise combining the active agent in the first container of a kit of the present invention and the vehicle of the second container of the kit of the present invention to form a therapeutically effective pharmaceutical preparation, and causing a therapeutically effective dose of the pharmaceutical preparation to be delivered to the patient.

A “receptacle” is any container or means for containing an active agent from which a patient receives a dose of an active agent. A receptacle can be a part of the kit, or it can be a separate item. A receptacle can be, for example, a spoon, a cap, a container of the kit, dosage measuring container such as a medicine dropper, a component of the kit that is adapted for delivering an asthma medication, or any other suitable means.

“Deliver” means to cause to be ingested or consumed by a patient. “Deliver” encompasses the use of any receptacle, including delivery from the kit or a container of the kit. “Deliver” encompasses self-delivery by a patient or delivery brought about by a health care professional.

A “dose” is an amount of a composition that is delivered during an administration. An “administration” is an occasion during which one or more doses are delivered to a patient. A dose can comprise one or more actual deliveries of the composition, for example one or more spoonfuls. A dose can also comprise one or more supplies of the composition. A dose can be up to or at least about 5 mL, up to or at least about 10 mL, up to or at least about 15 mL, up to or at least about 20 mL, up to or at least about 50 mL, up to or at least about 100 mL, or more.

A “supply” is the amount of a composition that is prepared when a pharmacist or other person combines the contents of one container of a kit with the contents of a second container of a kit. A supply can be up to or at least about 5 mL, up to or at least about 10 mL, up to or at least about 15 mL, up to or at least about 20 mL, up to or at least about 50 mL, up to or at least about 100 mL, up to or at least about 200 mL, up to or at least about 250 mL, up to or at least about 500 mL, or more. A “pharmaceutical preparation,” which may also be referred to as a “prepared composition,” can comprise one or more supplies.

The following examples are further illustrative of the present invention, but are not to be construed to limit the scope of the present invention.

### EXAMPLES

### Example 1

### Formulations

### Example 1A

### Oral Solution 4 mg/mL

The oral solution was prepared as follows: the active pharmaceutical ingredient (API) was dissolved in anhydrous ethanol and diluted with medium chain triglyceride (MCT) oil containing antioxidants, to form the composition presented in Table 1A, Solution 1. The API in this example was montelukast sodium, but other APIs may be replaced in appropriate amounts. The compositions of two example oral solutions are presented in Table 1A.

TABLE 1A
Oral solutions, 4 mg/mL

| | | | |
|---|---|---|---|
| Ingredient | Function | % w/w | Range |

Solution 1:

| | | | |
|---|---|---|---|
| MCT Oil, Miglyol 812 | Carrier vehicle | 94.585 | 5%-99.6% |
| Ethyl Alcohol | Solvent | 5.00 | 1%-95% |
| Anhydrous | | | |
| Butylated | Antioxidant | 0.01 | 0.0002%-1% |
| Hydroxyanisole | | | |
| (BHA) | | | |
| Butylated | Antioxidant | 0.01 | 0.0009%-1% |
| Hydroxytoluene | | | |
| (BHT) | | | |
| Montelukast Sodium | Active Agent | 0.395 | 0.01%-2% |

Solution 2 represents the amounts of each component
if a dispersing agent is used.

| | | | |
|---|---|---|---|
| MCT Oil, Miglyol 812 | Carrier vehicle | 91.585 | 5%-99.6% |
| Ethyl Alcohol | Solvent | 5.00 | 1%-95% |
| Anhydrous | | | |
| Butylated | Antioxidant | 0.01 | 0.0002%-1% |
| Hydroxyanisole | | | |
| (BHA) | | | |
| Butylated | Antioxidant | 0.01 | 0.0009%-1% |
| Hydroxytoluene | | | |
| (BHT) | | | |
| Montelukast Sodium | Active Agent | 0.395 | 0.01%-2% |
| Syloid 244 FP | Dispersing agent | 3.00 | 0.1%-20% |
| Silica gel | | | |

In the above-described formulation, stress studies have demonstrated that a formulation stability of, for example, about 10 weeks, 6 months, 1 year, 2 years or longer at room temperature can be achieved. Confirmed stability enhancers include protection from UV/ambient light, and saturation and storage under inert gas.

### Example 1A.1

### Oral Solution of Uncoated Active Agent in MCT Oil in a Light-Protective Kit

A volume sufficient for multiple doses of the preparation of Example 1A is packaged in a kit comprising a resealable light-protective container.

### Example 1B

### Oral Suspension 4 mg/mL

The oral suspension was prepared as follows: the API was combined with MCT oil containing antioxidants and silica gel.

The composition of the oral suspension is presented in Table 1B.

TABLE 1B
Oral suspension, 4 mg/mL.

| Ingredient | Function | % w/w | Range |
|---|---|---|---|
| MCT Oil, Miglyol 812 | Carrier vehicle | 98.585 | 5%-99.6% |
| Syloid 244 FP | Dispersing agent | 1.00 | 0.1%-20% |
| Silica gel | | | |
| Butylated | Antioxidant | 0.01 | 0.0002%-1% |
| Hydroxyanisole | | | |
| (BHA) | | | |
| Butylated | Antioxidant | 0.01 | 0.0009%-1% |
| Hydroxytoluene | | | |
| (BHT) | | | |
| Montelukast Sodium | Active agent | 0.395 | 0.01%-2% |

### Example 1B.1

### Oral Suspension of coated Granules in MCT Oil

The suspension is prepared as described in Example 1B using coated granules.

### Example 1C

### Oral Granules 4 mg/g

An oral granule composition was prepared as follows: the active agent was blended with granular mannitol to make a powder blend. The powder blend was then granulated with a solution of hydroxypropyl cellulose in anhydrous ethanol. The granulation was dried and sized to appropriate particle size. The composition of the oral granules is presented in Table 1C.

TABLE 1C
Oral granules, 4 mg/g

| Ingredient | Function | % w/w | Range |
|---|---|---|---|
| Mannitol granular | Carrier vehicle | 30.0 | 1%-99% |
| Mannitol Spray dried | Carrier vehicle | 64.88 | 10%-99% |
| Syloid 244 FP Silica gel | Dispersing agent | 3.00 | 0.1%-20% |
| Hydroxypropyl cellulose, | Binder, Disintegrant | 1.70 | 0.1%-99% |
| KLUCEL LF | | | |
| Montelukast sodium | Active agent | 0.420 | 0.01%-2% |

The reconstituted formulation was prepared by suspending the granules in MCT oil to prepare a final composition with active agent concentration of about 4 mg/mL. Stability studies indicate that the degradation rate is comparable to, and may be less than, that of the oral solution.

The shelf life of the granule formulation was extended by coating the granules with special coating materials, including enteric coating.

The granule formulations discussed above may be coated and conjugated to produce a controlled/slow/extended release drug formulation. This permits the drug to be administered once in a given 24 hour period, offering significant improvement in quality of life for the patient and effective dosing by the caregiver.

### Example 1C.1

### Oral Suspension of Uncoated Granules in Light-Protective Kit

Granules of the active agent prepared according to Example 1C are packaged in one container of a light-protective kit. The vehicle includes the antioxidants BHA and BHT and is packaged in a second container. The contents of the containers are combined.

### Example 1C.2

### Oral Suspension of Coated Granulesn Light-Protective Kit

The preparation of Example 1C.1 is prepared using coated granules and packaged in a light-protective kit.

### Example 1C.3

### Oral Suspension of Coated Active Agent in Aqueous Solution

A unit dosage of the active agent granules of Example 1C are combined with a liquid or foodstuff by the patient and consumed.

### Example 1C.4

### Oral Suspension of Coated Active Agent in Aqueous Solution in a Light-Protective Kit

The suspension prepared according to Example 1C.3 is packaged in a light-protective kit.

### Example 1C.5

### Oral Granules of Coated Active Agent in a Light-Protective Kit

Granules prepared according to Example 1C are coated and packaged in a light-protective kit.

### Example 2

### Analytical Development Studies

### Example 2A

### Stability Studies

Stability studies were carried out in various formulations to demonstrate extended shelf life for three formulations: 1) anhydrous oral solution; 2) anhydrous oral suspension; and 3) oral granules. The studies demonstrate an extended shelf life for the active agent in the inventive formulations, for example of about 10 weeks. When preparing the formulations for the study, no precautions were taken against active agent degradation due to UV light. Therefore, longer shelf life can be achieved if such precautions are taken.

The oxidative degradation of the exemplary active agent montelukast sodium was apparent when comparing the initial impurities for the oral solution, oral solution and oral granules. Total impurities in the granules were 1.87% as compared to the 0.86% in the oral solution. Sulfoxide, the major degradant, was 1.5 times higher in the oral granules.

The oral solution was stable. A low level of active degradation was observed for the solution. The longer preparation time of the oral granules under ambient conditions may have contributed to the greater degradation observed. Table 2 provides a detailed summary of the study.

TABLE 2
SCREEN STABILITY STUDY OF 3 FORMULATION TYPES; SOLUTION,
SUSPENSION AND GRANULES

| | | | | | | | |
|---|---|---|---|---|---|---|---|
| | Active | | Cis Isomer | Acid 2 | Acid 4 | Individual | Total |
| Stability | Agent Assay | Degradant | Degradant | Degradant | Degradant | Unknown | Impurities |
| Timepoint | (%) | (%) | (%) | (%) | (%) | Imps (%) | (%) |

ROOM TEMPERATURE STABILITY
SOLUTION (Lot: 58037)

| | | | | | | | |
|---|---|---|---|---|---|---|---|
| initial | 99.7 | 0.42 | ND | 0.11 | ND | ND | 0.53 |
| 2 months | 99.2 | 0.67 | ND | 0.14 | ND | 0.05 | 0.86 |

SUSPENSION (Lot: 58038)

| | | | | | | | |
|---|---|---|---|---|---|---|---|
| initial | 101.1 | 0.60 | ND | 0.17 | 0.13 | ND | 0.90 |
| 2 months | 99.2 | 0.96 | ND | 0.14 | ND | ND | 1.10 |

GRANULES (Lot: 58039)¹

| | | | | | | | |
|---|---|---|---|---|---|---|---|
| initial | 93.0 | 1.05 | 0.41 | 0.11 | 0.07 | 0.23 | 1.87 |
| 2 months | 89.9 | 1.56 | 0.35 | 0.12 | ND | 0.05 | 2.08 |

40° C. ACCELERATED STABILITY
SOLUTION (Lot: 58037)

| | | | | | | | |
|---|---|---|---|---|---|---|---|
| initial | 99.7 | 0.42 | ND | 0.11 | ND | ND | 0.53 |
| 4 weeks | 96.1 | 5.19 | ND | 0.17 | ND | 0.13 | 5.49 |

SUSPENSION (Lot: 58038)

| | | | | | | | |
|---|---|---|---|---|---|---|---|
| initial | 101.1 | 0.60 | ND | 0.17 | 0.13 | ND | 0.90 |
| 4 weeks | 95.0 | 7.22 | ND | 0.13 | ND | 0.07 | 7.42 |

GRANULES (Lot: 58039)¹

| | | | | | | | |
|---|---|---|---|---|---|---|---|
| initial | 93.0 | 1.05 | 0.41 | 0.11 | 0.07 | 0.23 | 1.87 |
| 4 weeks | 89.6 | 3.24 | 0.33 | 0.12 | ND | 0.06 | 3.75 |

50° C. ACCELERATED STABILITY
SOLUTION (Lot: 58037)

| | | | | | | | |
|---|---|---|---|---|---|---|---|
| initial | 99.7 | 0.42 | ND | 0.11 | ND | ND | 0.53 |
| 2 weeks* | 97.8 | 2.43 | ND | 0.17 | ND | ND | 2.60 |

SUSPENSION (Lot: 58038)

| | | | | | | | |
|---|---|---|---|---|---|---|---|
| initial | 99.7 | 0.42 | ND | 0.11 | ND | ND | 0.53 |
| 2 weeks* | 97.8 | 2.90 | ND | 0.12 | ND | ND | 3.02 |

GRANULES (Lot: 58039)¹

| | | | | | | | |
|---|---|---|---|---|---|---|---|
| initial | 99.7 | 0.42 | ND | 0.11 | ND | ND | 0.53 |
| 2 weeks* | 95.7 | 5.63* | ND | 0.14 | ND | ND | 0.14 |

¹Granules were studied for stability in the dry form; they were not dispersed or dissolved, for example into an MCT oil, in this study.
*(2-wk samples removed from 50° C. after 2 wks and kept at room temperature for 2 more wks)

### Example 2B

### Oral Solution Stress Study

The objective of the study was to identify the factors affecting oxidative degradation of an active agent in the context of the oral solution, as described above in Example 1. The following factors were also found to further extend stability of these formulations: 1) antioxidants: a) combined BHA (0.01%) and BHT (0.01%); and b) propyl gallate (0.02%). 2) exclusion of ambient light: yellow light was used when dispensing API. 3) exclusion of ambient atmosphere: used carbon dioxide headspace immediately upon transferring into capped glass vials.

API handling (weighing, dissolving etc.) was done under yellow light for all samples (except sample F3LX). Samples were tested initially immediately after preparation, represented by results for “bulk testing.” All samples were filled in 30 mL amber glass vials with Teflon closure. Purging and head-space saturation with CO₂ was applied to samples. Zero time tests were performed on prepared drug product samples on the following day. Prepared drug product samples were stored at 50° C. Follow up tests were conducted after 3 days and 7 days. All assays were conducted in duplicate.

Table 3 summarizes the details of test results for various studies using montelukast sodium as an exemplary labile active agent. The presence of BHA/BHT antioxidants combined with inert CO₂ headspace showed little degradation. If no CO₂ was used, the same samples performed comparably (see, e.g., sample F1YC as compared to sample F1YX). Without antioxidant the sulfoxide degradation was found to be 30 to 35% higher (see, e.g., samples F3YC and F3LX). The effect of ambient light was not pronounced. Sample F3LX, prepared under ambient light without CO₂ purging, compared similarly to F3YC, which was prepared under yellow light and stored under CO₂ headspace. All tests were carried out following exposure to 50° C. accelerated conditions.

TABLE 3
ANTIOXIDANT SELECTION (SOLUTION CONCEPT FORMULA)

| | | | | | | | | | |
|---|---|---|---|---|---|---|---|---|---|
| | | | | | | Unknown | Unknown | Unknown | |
| | | Sulfoxide | Cis Isomer | Acid 2 | Acid 4 | degradant | degradant | degradant | Total |
| Stability | | Degradant | Degradant | Degradant | Degradant | RRT 0.97 | RRT 1.08 | RRT 1.18 | Impurities |
| Timepoint | Assay (%) | (%) | (%) | (%) | (%) | (%) | (%) | (%) | (%) |

BHA, BHT + CO2 SPARGING (Lot: F1YC)

| Bulk testing | 102.3 | 0.21 | ND | 0.13 | ND | ND | ND | ND | 0.34 |
|---|---|---|---|---|---|---|---|---|---|
| Initial testing | 102.1 | 0.24 | ND | 0.12 | ND | ND | 0.07 | ND | 0.43 |
| 3 days | 102.0 | 0.52 | ND | 0.08 | ND | 0.10 | ND | ND | 0.70 |
| 7 days | 101.3 | 0.56 | ND | 0.11 | ND | 0.09 | ND | 0.06 | 0.82 |

BHA, BHT no CO2 (Lot: F1YX)

| | | | | | | | | | |
|---|---|---|---|---|---|---|---|---|---|
| Bulk testing | 101.9 | 0.24 | ND | 0.13 | ND | ND | 0.05 | ND | 0.42 |
| Initial testing | 101.4 | 0.32 | ND | 0.11 | ND | ND | 0.08 | ND | 0.51 |
| 3 days | 103.3 | 0.61 | ND | 0.08 | ND | ND | ND | ND | 0.69 |
| 7 days | 101.2 | 0.66 | ND | 0.12 | ND | ND | ND | ND | 0.78 |

PROPYL GALLATE + CO2 SPARGING (Lot: F2YC)

| | | | | | | | | | |
|---|---|---|---|---|---|---|---|---|---|
| Bulk testing | 97.4 | 0.22 | ND | 0.12 | ND | ND | ND | ND | 0.34 |
| Initial testing | 98.2 | 0.27 | ND | 0.11 | ND | ND | ND | ND | 0.38 |
| 3 days | 97.1 | 0.76 | ND | 0.15 | ND | ND | ND | ND | 0.91 |
| 7 days | 96.2 | 1.06 | ND | 0.17 | ND | ND | ND | ND | 1.23 |

PROPYL GALLATE no CO2 (Lot: F2YX)

| | | | | | | | | | |
|---|---|---|---|---|---|---|---|---|---|
| Bulk testing | 97.6 | 0.24 | ND | 0.12 | ND | ND | 0.05 | ND | 0.41 |
| Initial testing | 97.9 | 0.45 | ND | 0.11 | ND | ND | ND | ND | 0.56 |
| 3 days | 91.6 | 6.71 | ND | 0.08 | ND | ND | ND | ND | 6.79 |
| 7 days | 8.78 | 9.87 | ND | 0.10 | ND | ND | ND | 0.12 | 10.09 |

NO CHEMICAL ANTIOXIDANT + JUST CO2 SPARGING (Lot: F3YC)

| | | | | | | | | | |
|---|---|---|---|---|---|---|---|---|---|
| Bulk testing | 97.1 | 0.22 | ND | 0.12 | ND | ND | 0.08 | ND | 0.42 |
| Initial testing | 97.1 | 0.22 | ND | 0.10 | ND | ND | ND | ND | 0.32 |
| 3 days | 97.1 | 0.61 | ND | 0.08 | ND | ND | ND | ND | 0.69 |
| 7 days | 96.7 | 0.73 | ND | 0.28 | ND | ND | ND | 0.07 | 1.08 |

NO CHEMICAL ANTIOXIDANT NOR CO2 SPARGING (Lot: F3YX)

| | | | | | | | | | |
|---|---|---|---|---|---|---|---|---|---|
| Bulk testing | 96.6 | 0.21 | ND | 0.12 | ND | ND | 0.05 | ND | 0.38 |
| Initial testing | 98.0 | 0.32 | ND | 0.10 | ND | ND | ND | ND | 0.42 |
| 3 days | 97.1 | 0.70 | ND | 0.07 | ND | ND | ND | ND | 0.77 |
| 7 days | 96.6 | 0.76 | ND | 0.16 | ND | ND | ND | ND | 0.92 |

### Example 2c

### Oral Solution Extended Stability Testing

Oral solutions of montelukast sodium were tested for stability after storage at room temperature for approximately one year. The solutions were stored for three days at 50° C. prior to the one year room temperature storage period.

Table 4 summarizes the results. All samples other than F2YX (propyl gallate, no CO₂ sparging) and PD-213-38 (no antioxidant, no ambient light, 0.1840 g/g active in ethanol) were stable over the testing period.

TABLE 4
MONTELUKAST ORAL SOLUTION
ONE-YEAR STABILITY

| | | % LC of |
|---|---|---|
| Code | Description | Montelukast |
| F1YX | F1: Antioxidants (BHA (0.01%) + BHT | 99.7 |
| | (0.01%) | |
| | X: No CO₂ gas used. | |
| | Y Sample processed and filled under ambient | |
| | light (yellow) | |
| F2YX | F2: Antioxidant propyl gallate | 88.9 |
| | X: No CO₂ gas used. | |
| | Y Sample processed and filled under ambient | |
| | light (yellow) | |
| F3LX | F3 No Antioxidant (control) | 102.6 |
| | L Sample processed and filled under ambient | |
| | light (white) | |
| | X: No CO₂ gas used | |
| F1YC | F1: Antioxidants (BHA (0.01%) + BHT | 101.0 |
| | (0.01%) | |
| | C: 20 g filled in amber bottle, topped up | |
| | with CO₂ | |
| | Y: Sample processed and filled under ambient | |
| | light (yellow) | |
| F2YC | F2: Antioxidant propyl gallate | 101.0 |
| | C: 20 g filled in amber bottle, topped up | |
| | with CO₂ | |
| | Y: Sample processed and filled under ambient | |
| | light (yellow) | |
| F3YC | F3 No Antioxidant (control) | 99.1 |
| | C: 20 g filled in amber bottle, topped up | |
| | with CO₂ | |
| | Y: Sample processed and filled under ambient | |
| | light (yellow) | |
| PD213-38 | PD-213-38: No antioxidant or ambient light, | 80.8 |
| | Dissolved concentration of Active in ethanol | |
| | (0.1840 g/g) | |
| PD213-11 | PD-213-11: No antioxidant or ambient light, | 100.6 |
| | Dissolved concentration of Active in ethanol | |
| | (0.0894 g/g) | |

### Example 3

### Stability Testing for Other Active Agents

Other active agents are tested for stability in the same vehicles. The formulations have much less breakdown of active agents than aqueous formulations.

### Example 4

### Enhanced Stability in Aqueous Blends

Some compositions contain components other than the active agent that are photo-labile, hydrolysis-labile, oxidation-labile, or thermo-labile. For example, some compositions comprise an agent that neutralizes agents that would render the active agent ineffective. Such an agent can be referred to as a neutralizing agent. For example, an amoxicillin preparation may also include clavulanic acid as a neutralizing agent.

The compositions of the present invention have better stability than aqueous solutions, with respect to the active agents, neutralizing agents, or other vehicle components. To demonstrate this, samples of some embodiments of the present invention are prepared at neutralizing agent concentrations of 15 mg/5 mL, 35 mg/5 mL and 90 mg/5 mL. Each sample is dispersed in a vehicle of medium chain triglycerides and water. Furthermore, the sample preparation is repeated with the addition of an active agent at 250 mg/5 mL. Table 5 shows the ratios of MCT:water that are used in each sample prepared.

TABLE 5
RATIOS OF MCT TO WATER USED IN FORMULATIONS

| | Neutralizing agent | | |
|---|---|---|---|
| MCT:Water | 15 mg | 35 mg | 90 mg |
| (Diluent) | (Ratio ~17) | (Ratio ~7) | (Ratio ~3) |
| 1:3 | No active agent | No active agent | No active agent |
| (pH per USP | Active agent | Active agent | Active agent |
| between 3.8 to 6.6) | 250 mg/5 mL | 250 mg/5 mL | 250 mg/5 Ml |
| 1:1 | No active agent | No active agent | No active agent |
| (pH per USP | Active agent | Active agent | Active agent |
| between 3.8 to 6.6) | 250 mg/5 mL | 250 mg/5 mL | 250 mg/5 mL |
| 3:1 | No active agent | No active agent | No active agent |
| (pH per USP | Active agent | Active agent | Active agent |
| between 3.8 to 6.6) | 250 mg/5 mL | 250 mg/5 mL | 250 mg/5 mL |
| 100% MCT | No active agent | No active agent | No active agent |
| | Active agent | Active agent | Active agent |
| | 250 mg/5 mL | 250 mg/5 mL | 250 mg/5 mL |

The pH of the samples is controlled so that the mixture of active agent and neutralizing agent have a pH of about 3.8 to about 6.6. Solutions of the pure active agent tested (concentration of 2 mg/ml) have an optimum pH range of about 3.8 to about 6.0 and solutions of the pure neutralizing agent tested (1% solution) have an optimum pH range of about 3.8 to about 8.0.

These samples are stored at both a temperature of about 20° C. to about 25° C. and under refrigeration (about 4° C.) and are tested for pH and the amount of active agent and neutralizing agent. The samples to be tested for pH and the amount of active agent and neutralizing agent at the following time points: 0 day (day of reconstitution), 5 day, 10 day, 15 day, 1 month, 2 months and 3 months after reconstitution.

The pH is measured using any technique known to one of skill in the art. For example, one can use the methods described in the U.S. Pharmacopoeia.

The amounts of active agent and neutralizing agent are measured using HPLC or other assay capable of measuring the amount of active agent and neutralizing agent. Quantification of the compounds by HPLC is used to determine the unknown concentration of both active agent and neutralizing agent in the samples. The samples are injected (about 10-20 μl) into a liquid chromatograph equipped with a 210-nm detector and a 4-mm×30-cm column that contains 3- to 10-μm packing L1. See U.S. Pharmacopoeia. The peaks of the sample are compared to the injection of a series of known concentrations of the standard compound solution (e.g., about 10 μl of active agent and/or about 20μl of neutralizing agent) onto the HPLC for detection. The chromatograph of these known concentrations gives a series of peaks that correlate to the concentration of the samples that are injected. The chromatogram is recorded and the responses for the major peaks are measured. One of skill in the art can then determine the percentage of neutralizing agent or active agent in the solution using the methods described in the U.S. Pharmacopoeia or other equivalent method.

Furthermore, for in vivo studies testing the stability of the samples, high-performance liquid chromatographic methods using ultraviolet detection at 220 nm can be used for the simultaneous determination of active agent and neutralizing agent in human or dog plasma. See Hoizey et al., J Pharm Biomed Anal. 15;30(3):661-6 (2002); Choi et al., J Pharm Biomed Anal. 1;35(1):221-31 (2004).

As one of skill in the art will appreciate, the stability of the composition can be tested using methods other than HPLC. For example, bioassays and spectrophotometric methods could be used in place of HPLC or in addition to HPLC to determine the stability of the composition. Thus, testing using zone of inhibition assays, cell proliferation assays (e.g., using calorimetric dyes), and other equivalent methods are also encompassed by the present invention.

### Example 5

### Preparation of Formulation

The formulations in Table 6A-6D depict embodiments of the present invention. These formulations can be prepared by using the following exemplary methods.

The formulation depicted in Table 6A can be prepared by adding sorbitan monostearate and BHT to medium chain triglycerides to form a mixture, heating the mixture to 55° C., and then mixing the mixture until the components have dissolved. The resulting oil suspension is then cooled to a temperature of about 20° C. to about 25° C.

Next, the active agent, optional neutralizing agent/silicon dioxide blend (1:1 blend), silicon dioxide (63FP), and colloidal silicon dioxide are weighed into the amounts listed in Table 6A and then mixed in a container, such as, a glass bottle to form a dry blend. The oil suspension is then added to the container. The container is shaken to suspend the dry blend in the oil suspension.

TABLE 6A

| No. | Ingredient | Amount for 100 g |
|---|---|---|
| 1 | Active Agent | 3.05 |
| 2 | (Optional) neutralizing | 1.66 |
| | agent/silicon dioxide (1:1 | |
| | blend) | |
| 3 | Silicon dioxide 63FP | 1.05 |
| 4 | Colloidal silicon dioxide | 0.05 |
| 5 | Medium chain triglycerides | 93.98 |
| | (Myritol 318PH) | |
| 6 | Sorbitan monostearate | 0.2 |
| 7 | Butylated hydroxytoluene | 0.01 |
| | (BHT) | |

The formulation depicted in Table 6B can be prepared by adding sorbitan monostearate and BHT to medium chain triglycerides to form a mixture, heating the mixture to 55° C., and then mixing the mixture until the components have dissolved. The resulting oil suspension is then cooled to a temperature of about 20° C. to about 25° C.

Next, the active agent, optional neutralizing agent/silicon dioxide blend (1:1 blend), silicon dioxide (244FP), and colloidal silicon dioxide are weighed into the amounts listed in Table 6B and then mixed in a container, such as, a glass bottle to form a dry blend. The oil suspension is then added to the container. The container is shaken to suspend the dry blend in the oil suspension.

TABLE 6B

| No. | Ingredient | Amount for 100 g |
|---|---|---|
| 1 | Active agent | 3.05 |
| 2 | (Optional) Neutralizing | 1.66 |
| | agent/silicon dioxide (1:1 | |
| | blend) | |
| 3 | Silicon dioxide 244FP | 1.05 |
| 4 | Colloidal silicon dioxide | 0.05 |
| 5 | Medium chain triglycerides | 93.98 |
| | (Myritol 318PH) | |
| 6 | Sorbitan monostearate | 0.2 |
| 7 | Butylated hydroxytoluene | 0.01 |
| | (BHT) | |

The formulation depicted in Table 6C can be prepared by adding sorbitan monostearate and BHT to medium chain triglycerides to form a mixture, heating the mixture to 55° C., and then mixing the mixture until the components have dissolved. The resulting oil suspension is then cooled to a temperature of about 20° C. to about 25° C.

Next, the active agent, optional neutralizing agent/silicon dioxide blend (1:1 blend), silicon dioxide (72FP), and colloidal silicon dioxide are weighed into the amounts listed in Table 6C and then mixed in a container, such as a glass bottle, to form a dry blend. The oil suspension is then added to the container. The container is shaken to suspend the dry blend in the oil suspension.

TABLE 6C

| No. | Ingredient | Amount for 100 g |
|---|---|---|
| 1 | Active agent | 3.05 |
| 2 | (Optional) Neutralizing | 1.66 |
| | agent/silicon dioxide (1:1 | |
| | blend) | |
| 3 | Silicon dioxide 72FP | 1.05 |
| 4 | Colloidal silicon dioxide | 0.05 |
| 5 | Medium chain triglycerides | 93.98 |
| | (Myritol 318PH) | |
| 6 | Sorbitan monostearate | 0.2 |
| 7 | Butylated hydroxytoluene | 0.01 |
| | (BHT) | |

The formulation depicted in Table 6D can be prepared by adding sorbitan monostearate, BHA, and BHT to medium chain triglycerides to form a mixture, heating the mixture to 55° C., and then mixing the mixture until the components have dissolved. The resulting oil suspension is then cooled to a temperature of about 20° C. to about 25° C.

Next, the active agent, optional neutralizing agent/silicon dioxide blend (1:1 blend), silicon dioxide (63FP), and colloidal silicon dioxide are weighed into the amounts listed in Table 6D and then mixed in a container, such as, a glass bottle to form a dry blend. The oil suspension is then added to the container. The container is shaken to suspend the dry blend in the oil suspension.

TABLE 6D

| No. | Ingredient | Amount for 50 g |
|---|---|---|
| 1 | Active agent | 3.05 |
| 2 | (Optional) Neutralizing | 1.66 |
| | agent/silicon dioxide (1:1 | |
| | blend) | |
| 3 | Silicon dioxide 63FP | 0.525 |
| 4 | Colloidal silicon dioxide | 0.025 |
| 5 | Medium chain triglycerides | 47.00 |
| | (Myritol 318PH) | |
| 6 | Sorbitan monostearate | 0.1 |
| 7 | Butylated hydroxyanisole | 0.005 |
| | (BHA) | |
| 8 | Butylated hydroxytoluene | 0.005 |
| | (BHT) | |

### Example 7

### Water Content of MCT Oils

The following tests were conducted to determine the water content of un-desiccated MCT oils. Six samples of Miglyol 812 MCT Oil were titrated using Karl Fischer titration. Samples weight was varied from 5 to 9 grams, with associated change in titrant volume from approximately 1.5 ml to 2.8 ml. The results are presented in Table 7 below

TABLE 7
WATER CONTENT OF MIGLYOL 812

| Sample | Sample weight (g) | % Water |
|---|---|---|
| 1 | 9.3179 | 0.0266 |
| 2 | 9.4182 | 0.0257 |
| 3 | 9.4395 | 0.0274 |
| 4 | 6.2447 | 0.0261 |
| 5 | 5.1917 | 0.0293 |
| 6 | 5.8076 | 0.0297 |

Average: 0.0275%
% RSD: 6.1

The accuracy of the above-described test was verified as follows. Two portions of oil, about 70 g each, were mixed with water to bring total water to the level 0.1% and 0.4%, respectively. The oil-water mixtures were then stirred for 3 hours to obtain homogeneous emulsions. The samples were then analyzed in triplicate and recovery calculated. The results presented in Table 8 below:

TABLE 8
WATER RECOVERY

| | | Water detected | % Recovery |
|---|---|---|---|
| Water added | Sample | (% Total) | (Average of 3) |
| 0.0712% | 1 | 0.1034 | 106.9% |
| | 2 | 0.1028 | |
| | 3 | 0.1049 | |
| 0.4032% | 1 | 0.4013 | 94.3% |
| | 2 | 0.3985 | |
| | 3 | 0.4236 | |

Thus, the Karl Fischer method for determining the water content of MCT oil provided 10% precision on the level of 0.03%, and 6% accuracy within the range from about 0.1% to about 0.4% water content.

These examples illustrate possible embodiments of the present invention. As one of skill in the art will appreciate, because of the versatility of the compositions, kits, and methods of using the compositions disclosed herein, the compositions, kits, and methods can be used in other similar ways to those described herein. Thus, while the invention has been particularly shown and described with reference to some embodiments thereof, it will be understood by those skilled in the art that they have been presented by way of example only, and not limitation, and various changes in form and details can be made therein without departing from the spirit and scope of the invention. Therefore, the breadth and scope of the present invention should not be limited by any of the above-described exemplary embodiments, but should be defined only in accordance with the following paragraphs and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents. U.S. application Ser. No. 11/902,925, filed Sep. 26, 2007, is also incorporated by reference in its entirety and for all purposes, including the right of priority.

1. A pharmaceutical composition for oral administration comprising an exceptionally labile active agent, a stabilizing vehicle comprising liquid triglycerides and a desiccant, wherein the composition is storage stable for an extended period without substantial degradation of the active agent.

2. The composition of claim 1, wherein the liquid triglycerides comprise medium chain triglycerides.

3. The composition of claim 1, wherein the active agent is in suspension in the vehicle.

4. The composition of claim 1, wherein the active agent is in solution in the vehicle.

5. The composition of claim 1, wherein the triglycerides have a water content of less than about 0.01%.

6. The composition of claim 1, wherein the active agent is selected from the group consisting of clindamycin, lansoprazole, alendronate, niaprazine, zafirlukast, pranlukast and montelukast.

7. The composition of claim 1, wherein the active agent is montelukast sodium.

8. The composition of claim 7, wherein the montelukast sodium concentration is about 0.395% (w/w).

9. The composition of claim 1, wherein the active agent is stable for at least about 1 year when stored at a temperature of about 20° C. to about 25° C.

10. A pharmaceutical composition comprising an exceptionally labile active agent in a stabilizing solid vehicle comprising a desiccant, wherein the composition is suitable for combining with a liquid vehicle comprising medium chain triglycerides.

11. The composition of claim 10, wherein the active agent is selected from the group consisting of clindamycin, lansoprazole, alendronate, niaprazine, zafirlukast, pranlukast and montelukast.

12. The composition of claim 10, wherein the active agent is montelukast sodium.

13. The composition of claim 10, wherein the desiccant comprises granular mannitol and/or spray dried mannitol.

14. The composition of claim 10, further comprising a silica gel.

15. The composition of claim 10, further comprising an enteric coating comprising a material selected from the group consisting of polyvinyl acetate phthalate, hydroxypropyl methylcellulose phthalate, a methacrylic acid-methacrylic acid ester copolymer, cellulose acetate trimellitate, carboxymethyl ethylcellulose, hydroxypropyl methylcellulose acetate succinate, a methacrylic acid-ethyl acrylate copolymer, a methacrylic acid-methyl methacrylate copolymer, a basic butylated methacrylate copolymer, an ammonio methacrylate copolymer, a poly(ethylacrylat-methylmethacrylat) dispersion, a methacrylic acid co-polymer, ethyl cellulose coating, and combinations thereof.

16. A reconstituted composition comprising the composition according to claim 10 and medium chain triglycerides.

17. The pharmaceutical composition of claim 16, further comprising an antioxidant.

18. A method of making the composition according to claim 4, comprising the steps of:
dissolving the exceptionally labile active agent in anhydrous ethanol to make a solution, and
diluting the solution with a vehicle comprising dry medium chain triglycerides and a desiccant,
thereby making the composition.

19. A method of making the composition according to claim 10, comprising the steps of:
blending the exceptionally labile active agent with granular mannitol to make a powder blend,
granulating the powder blend with a solution comprising hydroxypropylcellulose and anhydrous ethanol to make a granulation comprising particles, and
drying the granulation and sizing the particles.

20. A method of making a reconstituted composition comprising combining the composition according to claim 10 with medium chain triglycerides.

21. A method of treating an illness in a patient in need thereof comprising administering a therapeutically effective amount of the composition according to claim 1 to the patient orally, thereby treating the illness.

22. The method of claim 21, wherein the patient is a pediatric patient.

23. The method of claim 21, wherein the dose is in an amount of about 5 mL.

24. A method of treating an illness in a patient in need thereof comprising administering a therapeutically effective amount of the composition according to claim 16 to the patient orally, thereby treating the illness.

25. A kit comprising:
(a) a first container comprising a therapeutically effective amount of a solid composition comprising an exceptionally labile active agent and a desiccant, and
(b) a second container comprising a pharmaceutically acceptable liquid carrier comprising triglycerides, and an excipient, diluent or combination thereof, suitable for oral administration.

26. The kit of claim 25, wherein either or both of the first container and second container are light-protective containers.

27. The kit of claim 25, wherein either or both of the first container and second container are made of high density polyethylene.

28. The kit of claim 25, wherein the carrier further comprises an antioxidant.
